(19) United States
(12) Patent Application Publication
(10) Pub. No.: US 2010/0130875 A1
(43) Pub. Date: May 27, 2010

(54) BODY-WORN SYSTEM FOR MEASURING BLOOD PRESSURE

(75) Inventors: Matthew J. BANET, Del Mar, CA (US); Kenneth R. HUNT, Vista, CA (US); Henk VISSER, II, San Diego, CA (US)

Correspondence Address: WilmerHale/Triage Wireless, 60 State Street, Boston, MA, 02109, US

(73) Assignee: TRIAGE WIRELESS, INC., San Diego, CA (US)

(21) Appl. No.: 12/487,283

(22) Filed: Jun. 18, 2009

Related U.S. Application Data
(60) Provisional application No. 61/073,681, filed on Jun. 18, 2008.

(51) Int. Cl. A61B 5/021; A61B 5/02

(52) U.S. Cl. 600/490; 600/500

(57) ABSTRACT

A system for measuring a blood pressure value from a patient features a sensor configured to be worn on the patient's thumb. The sensor includes one or two light sources that emit optical radiation, and a photodetector that detects the optical radiation after it passes through a portion of a vessel (e.g. an artery or capillary) in the patient's thumb to generate a first time-dependent signal (e.g. a PPG waveform). In embodiments the sensor is made from a flexible material that wraps around a portion of the patient's thumb (e.g. the base) while leaving the thumb's tip uncovered. This configuration is less awkward than most finger-worn sensors, and allows the patient to comfortably go about their day-to-day activities (e.g. reading, eating) with little obstruction. The system also includes at least two electrodes that are configured to be worn on the patient's body and detect electrical signals that are processed by an electrical circuit to generate a second time-dependent signal (e.g. an ECG waveform).

This application claims the benefit of U.S. Provisional Application No. 61/073,681, filed Jun. 18, 2008, all of which is incorporated herein by reference.

### TECHNICAL FIELD

The present invention relates to medical devices for monitoring vital signs, e.g., blood pressure.

### BACKGROUND OF THE INVENTION

Pulse transit time (PTT), defined as the transit time for a pressure pulse launched by a heartbeat in a patient's arterial system, has been shown in a number of studies to correlate to both systolic and diastolic blood pressures. In these studies, PTT is typically measured with a conventional vital signs monitor that includes separate modules to determine both an electrocardiogram (ECG) and pulse oximetry. During a PTT measurement, multiple electrodes typically attach to a patient's chest to determine a time-dependent ECG component characterized by a sharp spike called the ‘QRS complex’.

The QRS complex indicates an initial depolarization of ventricles within the heart and, informally, marks the beginning of the heartbeat and a pressure pulse that follows. Pulse oximetry is typically measured with a bandage or clothespin-shaped sensor that attaches to a patient's finger, and includes optical systems operating in both the red and infrared spectral regions. A photodetector measures radiation emitted from the optical systems that transmits through the patient's finger. Other body sites, e.g., the ear, forehead, and nose, can be used in place of the finger. During a measurement, a microprocessor analyses both red and infrared radiation measured by the photodetector to determine the patient's blood oxygen saturation level and a time-dependent waveform called a photoplethysmograph ('PPG'). Time-dependent features of this waveform indicate both pulse rate and a volumetric absorbance change in an underlying artery caused by the propagating pressure pulse.

Typical PTT measurements determine the time separating a maximum point on the QRS complex, indicating the peak of ventricular depolarization, and a foot of the PPG waveform, indicating the time when a pressure pulse launched by the heartbeat reaches vasculature underneath the optical sensor. PTT depends primarily on arterial compliance, the propagation distance of the pressure pulse (which is closely approximated by the patient's arm length), and blood pressure. To account for patient-dependent properties, such as arterial compliance, PTT-based measurements of blood pressure are typically ‘calibrated’ using a conventional blood pressure cuff and oscillometry. Typically during the calibration process the blood pressure cuff is applied to the patient, used to make one or more blood pressure measurements, and then removed. Going forward, the calibration blood pressure measurements are used, along with a change in PTT, to determine the patient's blood pressure and blood pressure variability. PTT typically relates inversely to blood pressure, i.e., a decrease in PTT indicates an increase in blood pressure.

A number of issued U.S. Patents describe the relationship between PTT and blood pressure. For example, U.S. Pat. Nos. 5,316,008; 5,857,975; 5,865,755; and 5,649,543 each describe an apparatus that includes conventional sensors that measure ECG and PPG waveforms, which are then processed to determine PTT.

### SUMMARY OF THE INVENTION

In one aspect, a system for measuring a blood pressure value from a patient features a sensor configured to be worn on the patient's thumb. The sensor includes one or two light sources that emit optical radiation, and a photodetector that detects the optical radiation after it passes through a portion of a vessel (e.g. an artery or capillary) in the patient's thumb to generate a first time-dependent signal (e.g. a PPG waveform). In embodiments the sensor is made from a flexible material that wraps around a portion of the patient's thumb (e.g. the base) while leaving the thumb's tip uncovered. This configuration is less awkward than most finger-worn sensors, and allows the patient to comfortably go about their day-to-day activities (e.g. reading, eating) with little obstruction. The system also includes at least two electrodes that are configured to be worn on the patient's body and detect electrical signals that are processed by an electrical circuit to generate a second time-dependent signal (e.g. an ECG waveform).

A processing system worn on a portion of the patient's arm collectively process both the first and second time-dependent signals to determine the blood pressure value. It features a mechanical housing with a first input port that receives the first time-dependent signal, or a signal used to generate the first time-dependent signal, and a second input port that receives the second time-dependent signal, or a signal used to generate the second time-dependent signal. The first input port is located on one side portion of the housing, and the second input port is located on a second side portion that is opposite to the first side portion. This configuration minimizes cable clutter around the processing system: a first cable can run down the patient's wrist to the thumb-worn sensor, and a second cable can run up the patient's arm to chest-worn electrodes.

In embodiments, the processing system includes at least two separate portions, or compartments, both configured to be worn on separate portions of the patient's arm. A flexible housing can include both portions and securely wrap around the patient's arm. In embodiments, the first portion can include a processor (e.g. a microprocessor or microcontroller) programmed to process the first and second time-dependent signals to determine the blood pressure value. And the second portion can include a pneumatic system that features a pump, valve, and manifold, and generates a third time-dependent signal (e.g. a pressure waveform) representing pressure applied to the patient's arm. Typically the first and second portions connect to each other through a flexible cable. An armband that can be inflated by the pneumatic system attaches one or both portions to the patient's arm.

The processor can be programmed to process a time difference separating a first feature of the first time-dependent signal, and a second feature of the second time-dependent signal. This time difference can be processed by the composite technique to determine the blood pressure value. According to this technique, for example, the pneumatic system can supply a pressure to the patient's arm, and the processor is then programmed to process the first time-dependent signal in the presence of the supplied pressure (represented, e.g., by the third time-dependent signal) to determine a blood pressure calibration relating PTT to blood pressure. In one case, the processor processes the time difference in the presence of the supplied pressure to determine the blood pressure calibration according to the composite technique. In another case the processor processes a decrease in the amplitude of the first time-dependent signal to determine the blood pressure calibration.

In other embodiments the processing system includes a wireless transmitter that can transmit information to an external display.

The composite technique includes both pressure-dependent and pressure-free measurements. It is based on the discovery that PTT and the PPG waveform used to determine it are strongly modulated by an applied pressure. Two events occur as the pressure gradually increases to the patient's systolic pressure: 1) PTT increases in a non-linear manner once the applied pressure exceeds diastolic pressure; and 2) the magnitude of the PPG waveform's amplitude systematically decreases, typically in a linear manner, as the applied pressure approaches systolic pressure. The applied pressure gradually decreases blood flow and consequent blood pressure in the patient's arm, and therefore induces the pressure-dependent increase in PTT. Each of the resulting pairs of PTT/blood pressure readings measured during the period of applied pressure can be used as a calibration point. Moreover, when the applied pressure equals systolic blood pressure, the amplitude of the PPG waveform is completely eliminated, and PTT is no longer measurable. In total, analyzing both PTT and the PPG waveform's amplitude over a suitable range yields the patient's systolic and diastolic blood pressures. The composite technique measures systolic blood pressure directly; in contrast, conventional cuff-based systems based on the oscillometric technique measure this property indirectly, which is typically less accurate.

In addition, the composite technique can include an ‘intermediate’ pressure-dependent measurement wherein the armband is only partially inflated. This applies pressure to the patient's arm and partially decreases the amplitude of the PPG waveform in a time-dependent manner. The amplitude's pressure-dependent decrease can then be ‘fit’ with a numerical function to estimate the pressure at which the amplitude completely disappears, indicating systolic pressure.

For the pressure-dependent measurement, a small mechanical pump in the body sensor inflates the bladder to apply pressure to an underlying artery according to the pressure waveform. The armband is typically located on the patient's upper arm, proximal to the brachial artery, and time-dependent pressure is measured by an internal pressure sensor in the body sensor. The pressure sensor is typically an in-line Wheatstone bridge or strain gauge. The pressure waveform gradually ramps up in a mostly linear manner during inflation, and then rapidly deflates through a ‘bleeder valve’ during deflation. During inflation, mechanical pulsations corresponding to the patient's heartbeats couple into the bladder as the applied pressure approaches diastolic pressure.

The mechanical pulsations modulate the pressure waveform so that it includes a series of time-dependent oscillations. The oscillations are similar to those measured with an automated blood pressure cuff using oscillometry, only they are measured during inflation rather than deflation. They are processed as described below to determine mean arterial pressure, which is then used going forward in the pressure-free measurement. Specifically, the maximum amplitude of the pulsations corresponds to mean arterial pressure; measuring this property from the pressure waveform represents a direct measurement. Once determined, direct measurements of systolic and mean arterial pressure made during the pressure-dependent measurement are used to determine diastolic pressure using a numerical calculation, described in more detail below.

Pressure-free measurements immediately follow the pressure-dependent measurements, and are typically made by determining PTT with the same optical and electrical sensors used in the pressure-dependent measurements. Specifically, the body sensor processes PTT and other properties of the PPG waveform, along with the measurements of systolic, diastolic, and mean arterial pressure made during the pressure-dependent measurement, to determine blood pressure.

Advantages of the flexible body sensor include: i) a light-weight flexible form factor that easily conforms to a patient's arm; ii) a durable, sterile housing that protects internal electrical components from heat, moisture, and direct submersion in water; and, iii) simple, clutter-free wiring to the optical sensor and electrodes.

In addition to blood pressure, the body sensor measures heart rate and respiratory rate from components of the ECG waveform. These measurements are made using conventional algorithms. An optional pulse oximeter measures SpO2. The body sensor can also measure temperature (with a thermocouple); motion, posture, and activity level (with one or more accelerometers); and respiratory rate (with a chest-worn acoustic sensor or accelerometer integrated with one of the electrodes).

### BRIEF DESCRIPTION OF THE DRAWINGS

FIG. 1 shows a schematic drawing of a patient wearing a body sensor, in communication with an external monitor, and connected to an optical sensor and electrical sensors to measure blood pressure.

FIGS. 2A and 2B show schematic drawings of the optical sensor of FIG. 1 that is to be worn on a patient's thumb.

FIG. 3 shows a cross-sectional view the optical sensor of FIGS. 2A and 2B.

FIG. 4 shows a schematic view of the optical sensor of FIG. 3 worn on a patient's thumb.

FIGS. 5A and 5B show graphs of time-dependent PPG waveforms measured, respectively, from a patient's forearm and thumb measured with the optical sensor of FIGS. 2A and 2B.

FIGS. 6A and 6B show, respectively, schematic top-side and bottom-side views of the electrical components in the body sensor of FIG. 1.

FIG. 7 shows a schematic top view of the electrical components of FIGS. 6A and 6B.

FIG. 8 shows a schematic top view of a flexible housing used to enclose the electrical components of FIG. 7.

FIG. 9 is a cross-sectional view of the flexible housing of FIG. 8 with its armband wrapped around a patient's arm.

FIG. 10 shows a three-dimensional plan view of the monitor of FIG. 1.

### DETAILED DESCRIPTION

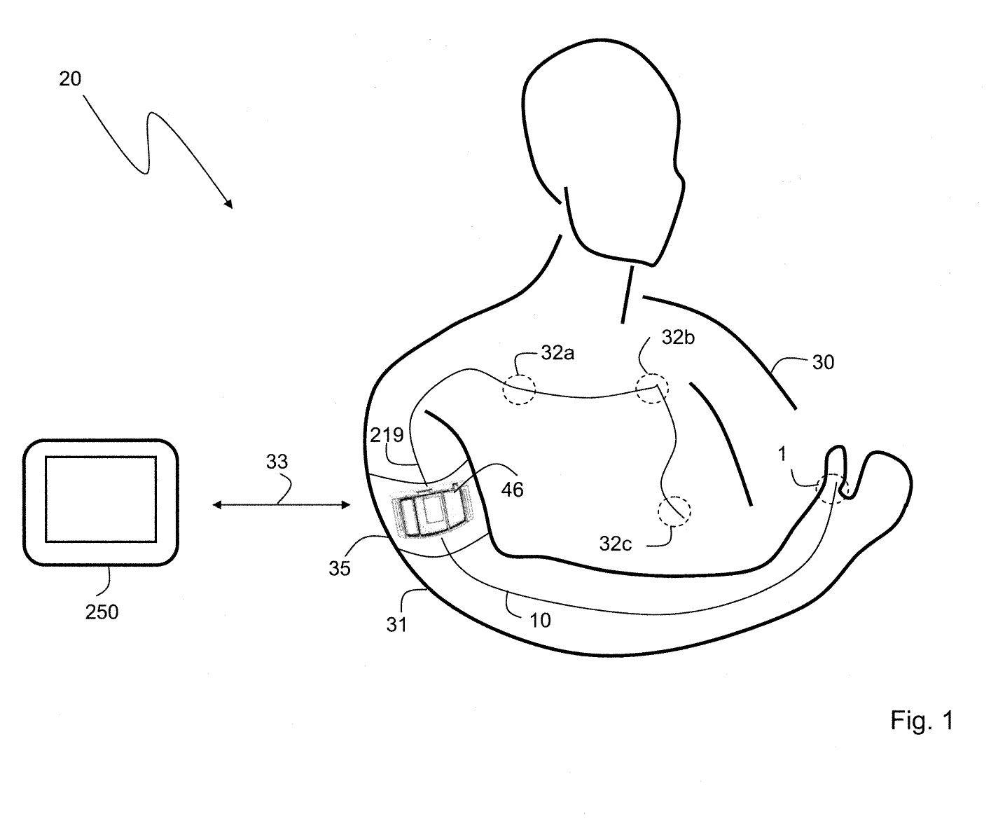

FIG. 1 shows a patient 30 wearing a system for measuring blood pressure 20 featuring a body sensor 46 connected to an optical sensor 1 worn on the patient's thumb and electrical sensors 32a-c (e.g. ECG electrodes) worn on the patient's chest. The body sensor 46 communicates wirelessly (as shown by the arrow 32) with a remote monitor 25. It attaches to the patient's arm 31 with an armband 35 similar to a blood pressure cuff. The ECG electrodes 32a-c adhere to the patient's chest in a standard Einthoven's triangle configuration, and connect to the body sensor 46 though a first cable 219. These electrodes 32a-c, in combination with a differential amplifying ECG circuit within the body sensor 46, measure an ECG waveform.

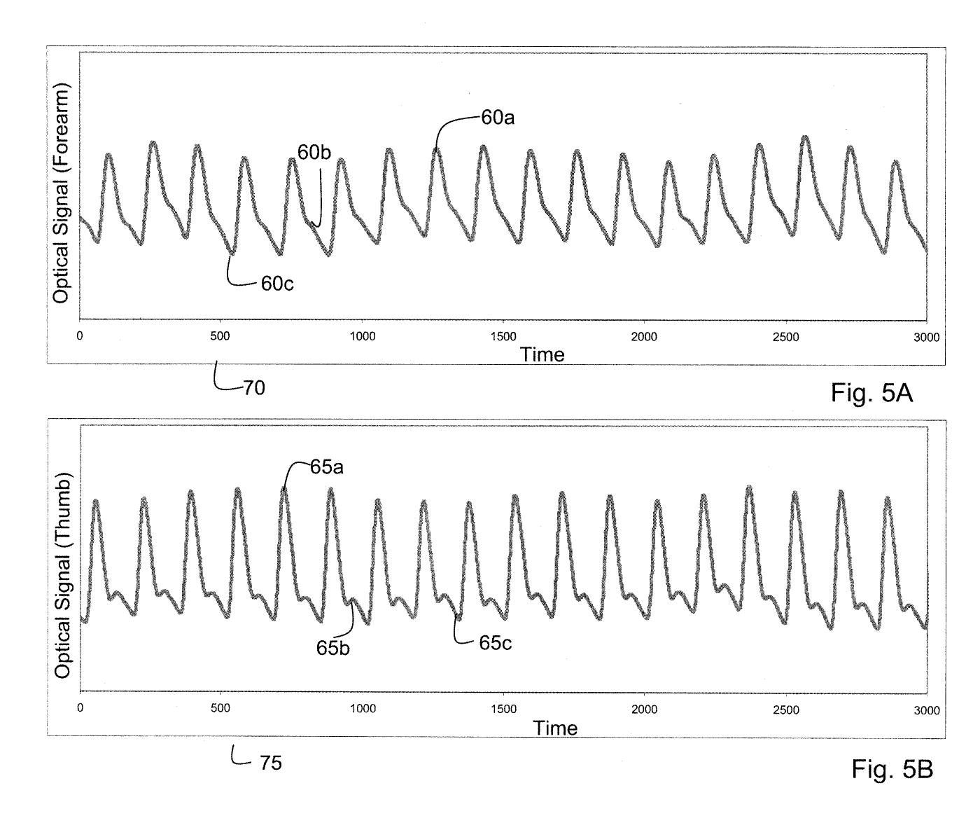

The optical sensor 1 wraps around the base of the patient's thumb with an adhesive band, and in combination with the body sensor 46 measures a PPG waveform similar to that indicated in graph 75 in FIG. 5B. During a pressure-dependent measurement, pneumatic components (i.e. a pump, valves, and pressure manifold) within a compartment in the body sensor 46 inflate a bladder within the armband 35, causing it to apply pressure to the patient's arm 31. The applied pressure has essentially no affect on the ECG waveform, but decreases the amplitude and delays the onset of pulses in the PPG waveform. A microprocessor in the body sensor 46 processes waveforms measured during the pressure-dependent measurement to ‘calibrate’ the measure for the particular patient 30 according to the composite technique. Subsequent pressure-free measurements use the calibration, along with a PTT determined from the PPG and ECG waveforms, to continuously determine the patient's blood pressure.

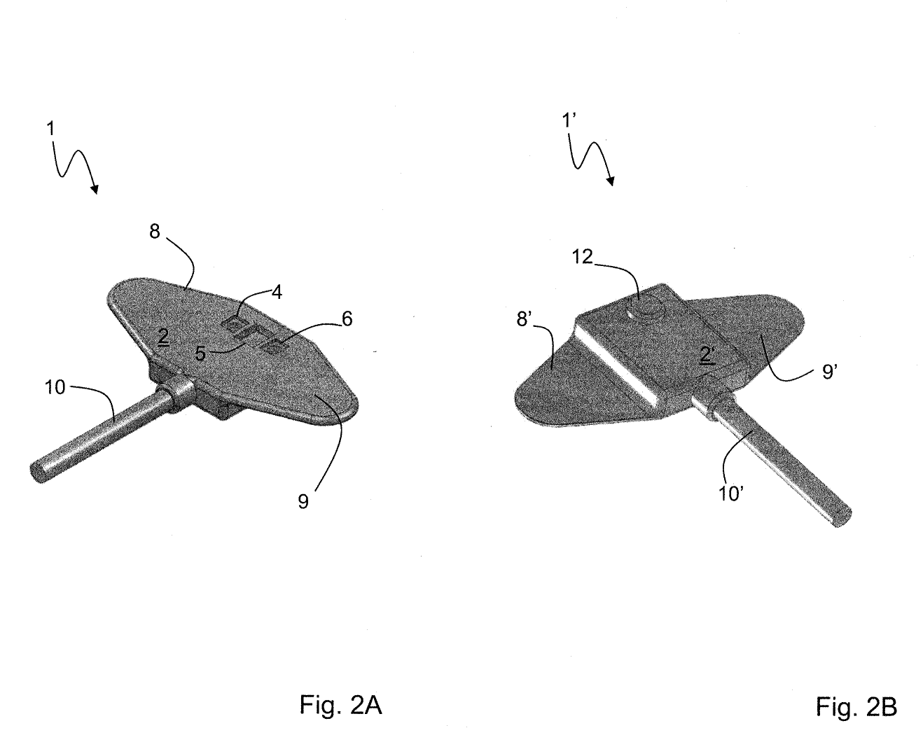

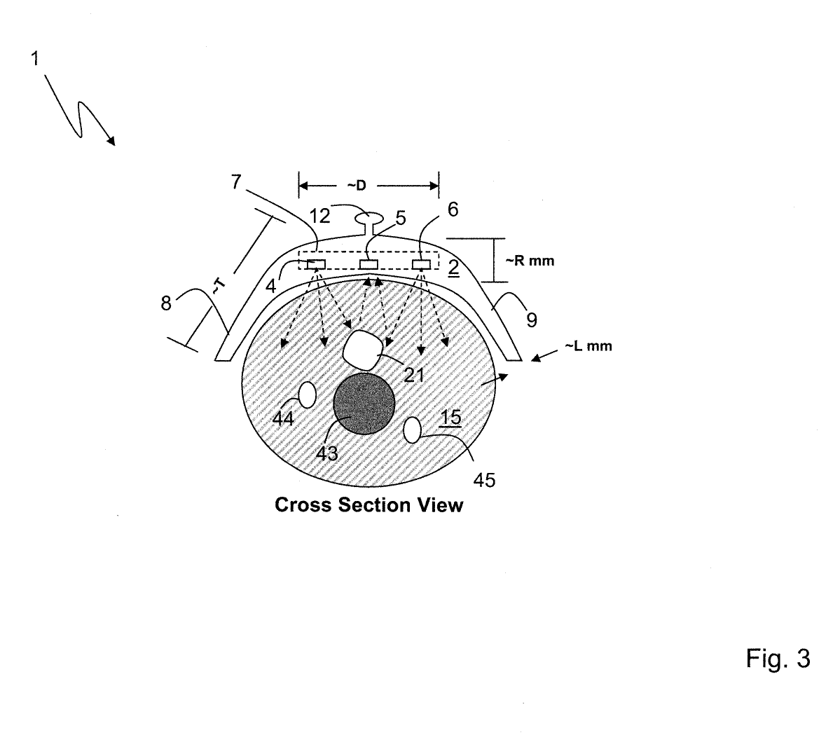

FIGS. 2A, 2B, and 3 show top, bottom, and cross-sectional views of the above-described optical sensor 1, 1′. It adheres to the patient's thumb with an adhesive wrap and connects to the body sensor 46 through a cable 10, 10′. The optical sensor 1, 1′ measures a PPG waveform from the patient during both pressure-dependent and pressure-free measurements. In the embodiment shown in FIGS. 2A and 2B, the optical sensor includes a single photodetector 5 between a pair of LEDs 4, 6. The LEDs typically operate in either the visible (e.g. 400-700 nm) or near-infrared (700-1000 nm) spectral regions. A flexible sensor housing 2, 2′ supports these optical components to make a measurement using either a reflection-mode or transmission-mode geometry. The optical components can be disposed in other configurations, e.g. the optical sensor 1, 1′ can include even more LEDs and photodetectors, or one or more separate optical modules, each including a single LED, photodetector, and analog amplifier. To make an optical measurement, the patient applies the flexible right flap 8, 8′ and left flap 9, 9′ of the flexible sensor housing 2, 2′, typically made up of black latex rubber or a comparable composite material, to the interior base of the thumb. The above-described system measures PPG waveforms for both the pressure-dependent and pressure-free measurements.

The following co-pending patent applications, the contents of which are fully incorporated herein by reference, describe the above-mentioned sensors and their use with the composite technique in more detail: 1) VITAL SIGN MONITOR FOR CUFFLESSLY MEASURING BLOOD PRESSURE USING A PULSE TRANSIT TIME CORRECTED FOR VASCULAR INDEX (U.S. Ser. No. 12/138,199, filed Jun. 12, 2008); and 2) VITAL SIGN MONITOR FOR MEASURING BLOOD PRESSURE USING OPTICAL, ELECTRICAL, AND PRESSURE WAVEFORMS (U.S. Ser. No. 12/138,194, filed Jun. 12, 2008).

To hold the optical sensor 1, 1′ in place, an extension or mushroom cap 12 provides a pressure point where an adhesive strip wraps around the patient's thumb, immobilizing the sensor to help reduce any motion-related artifacts and ambient noise generated from movement of the sensor. A thin flexible cable 10, 10′, roughly 42 cm to 54 cm in length, provides an electrical connection to the body sensor 46.

FIG. 3 shows a cross-sectional diagram and dimensions of the optical sensor 1 and optical radiation reflecting off the princeps pollics artery 21 in a patient's thumb 15. Both the flexible black latex rubber housing 2 and an internal circuit board 7 that supports the optical components 4, 5, 6 conform to the patient's thumb 15, allowing radiation to pass through the patient's skin and reflect around the bone 43, capillaries 44, 45, and artery 21 in the thumb 15. The flexible black latex rubber flaps 8, 9 are each approximately 12 mm in length each (represented by ˜T). The flexible circuit board 7 that supports the LEDS 4, 6, and photodetector 5 is embedded within the rubber housing 2, and is approximately 12 mm across (represented by ˜D). The combined height of the flexible housing 2 and body 7 is approximately 3 mm (represented by ˜R). Each flap 8, 9 has a thickness of approximately 1.5 mm (represented by ˜L).

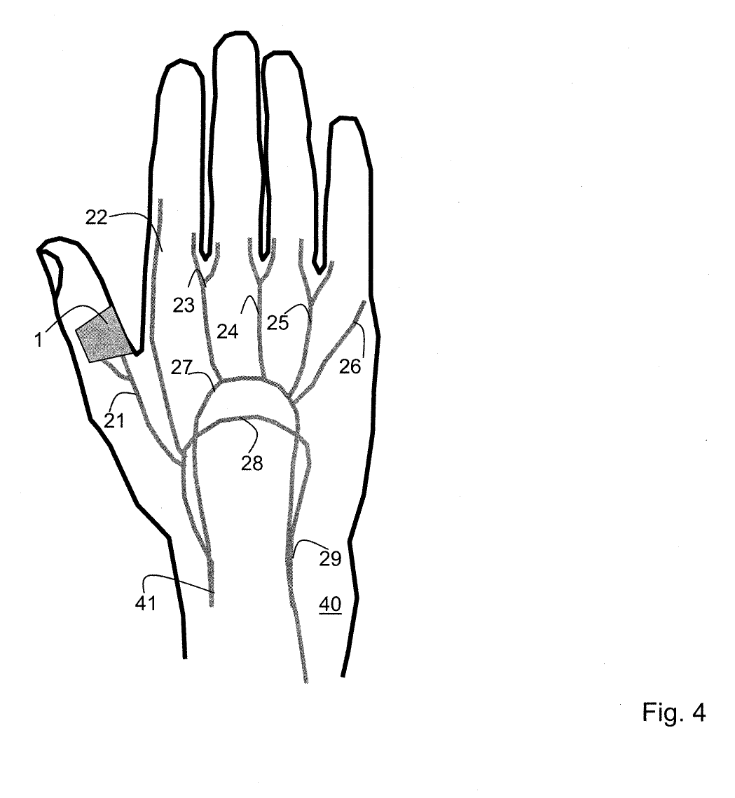

Referring to FIG. 4, with each heartbeat blood pumps through the patient's hand 40, starting from the ulnar 29 and radial 41 arteries, to deliver blood to patient's palmer arches 27, 28 and further distribute oxygenated blood to the digital arteries 22, 23, 24, 25, and 26 in each finger and thumb. Blood pressure, however, is typically strongest in the princeps pollics artery 21 of the thumb, which represents a direct extension of the radial artery 41. Placement of the sensor 1 on the lower inner portion of the thumb is therefore ideal to generate a PPG waveform that is: i) relatively free of motion-related artifacts; and, ii) of high signal strength due to the relatively strong blood pressure and good circulation within the princeps pollics artery.

Referring to FIG. 5B, the PPG waveform measured from the thumb in graph 75 shows a strong peak 65a, well-defined base 65c, and identifiable dichrotic notch 65b; each of these features is useful for generating accurate PTT-based blood pressure readings according to the composite technique. In contrast, the PPG waveform generated by measuring the patient's forearm 70 shows less-defined pulses, as shown in FIG. 5A. Signal quality from the pulse amplitudes measured in the forearm region tend to decrease gradually over time and have more rounded peaks 60a, less-defined bases 60c, and an almost non-existent dichotic notch 60b. These waveforms, in contrast to the PPG waveforms shown in FIG. 5B, tend to yield PTT-based blood pressure readings with relatively low accuracy.

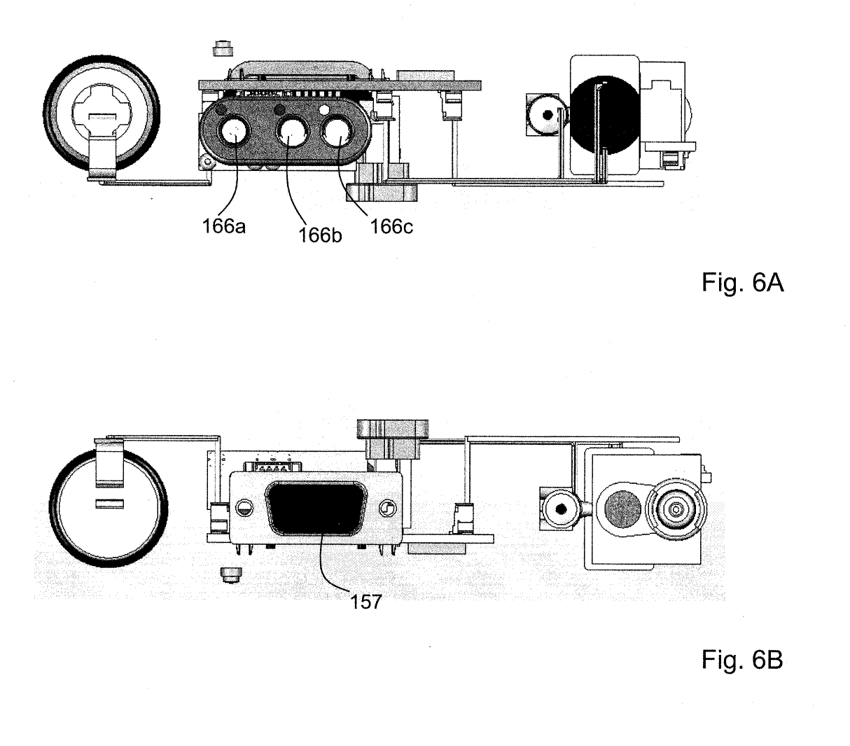

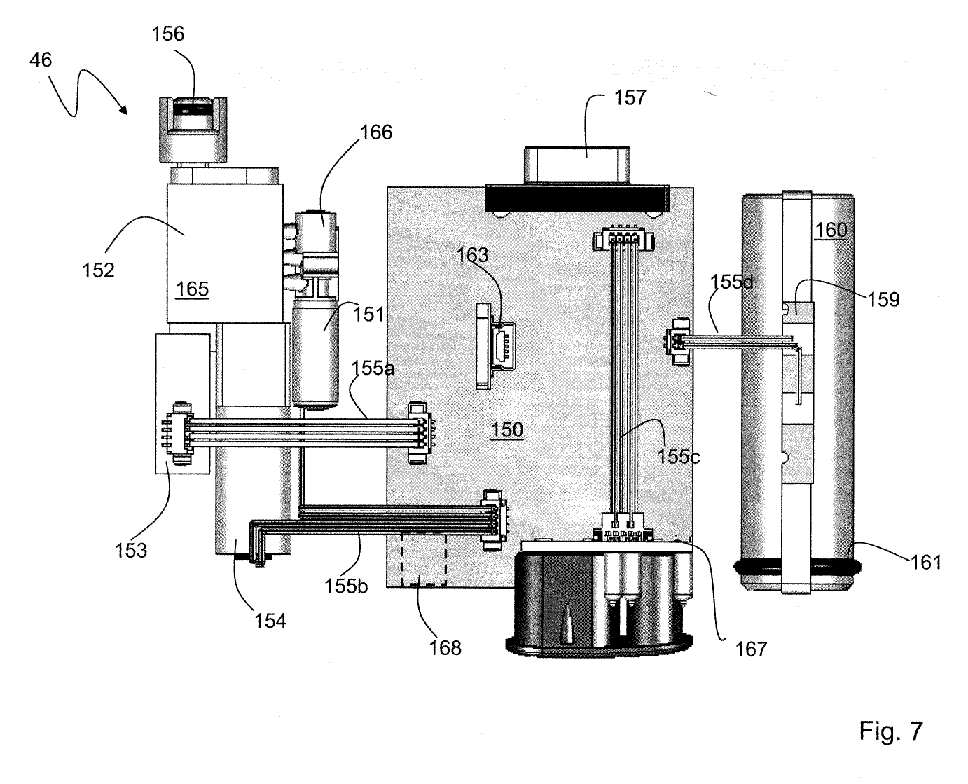

FIGS. 6A, 6B, and 7 show top-side, bottom-side, and top views of the body sensor 46 used to conduct the above-described measurements. The body sensor 46 features a single motherboard 150 connected to a battery component 160 and pneumatic component 165 through a series of flexible electrical wire harness cables 155a-d. In this way, the body sensor 46 is divided into three different compartmentalized components that, collectively, can easily wrap around the patient's arm. The motherboard 150 includes connectors 166a-c that connect through separate cables to the ECG electrodes worn on the patient's chest, and a DB-9 connector 157 that connects through the cable 10 to the optical sensor worn on the patient's thumb. The connectors 166a-c also make electrical connections to a defibrillation-protection circuit 167 that protects the internal electrical components from voltage spikes that occur during defibrillation. During both pressure-dependent and pressure-free measurements, these optical and electrical sensors measure signals that pass through the connectors 166a-c, 157 to discrete circuit components on the top-side and bottom-side of the motherboard 150.

The discrete components on the motherboard 150 include: i) analog circuitry for amplifying and filtering the time-dependent PPG and ECG waveforms; ii) an analog-to-digital converter for converting the time-dependent analog signals into digital waveforms; and iii) a microprocessor configured/programmed for processing the digital waveforms to determine blood pressure according to the composite technique, along with other vital signs, as described above.

To measure the pressure waveform during a pressure-dependent measurement, the pneumatic system 165 additionally includes a small mechanical pump 154 for inflating the bladder within the armband (shown in FIG. 1 as 35), and solenoid valves 151 for controlling the bladder's inflation and deflation rates. The pump 154 and solenoid valves 151 connect through a manifold 152 to a connector 156 that attaches through a tube (not shown in the figure) to the bladder within the armband, and additionally to a pressure sensor 153 that senses the pressure in the bladder. The solenoid valve 151 couples through the manifold 152 to a small, adjustable ‘bleeder’ valve 166 featuring a small hole that quickly releases pressure once a measurement is complete. Typically the solenoid valve 151 is closed as the pump 154 inflates the bladder. For measurements conducted during inflation, pulsations caused by the patient's heartbeats couple into the bladder as it inflates, and are mapped onto the pressure waveform. The pressure sensor 153 generates an analog pressure waveform, which is then digitized with the analog-to-digital converter described above, and finally filtered and processed to measure blood pressure during inflation. These blood pressure values are used to calibrate the PTT-based pressure-free measurements.

Alternatively, for measurements done on deflation, the pump 154 inflates the bladder to a pre-programmed pressure above the patient's systolic pressure. Once this pressure is reached, the microprocessor opens the solenoid valve 151, which couples to the ‘bleeder’ valve 166 adjusted to a setting that slowly releases pressure in the armband. During this deflation period, pulsations caused by the patient's heartbeat are coupled into the bladder and are mapped onto the pressure waveform, which is then measured by the digital pressure sensor, as described above. Once the microprocessor determines systolic, mean arterial and diastolic blood pressure, it opens the solenoid valve 151 to rapidly evacuate the pressure.

A rechargeable lithium ion battery 160 connects to a battery-protection circuit 159, which further connects through a harness 155d directly to the motherboard board 150 to power all the above-mentioned circuit components. The battery 160 also includes an O-ring 161 to ensure proper placement and stabilization. The motherboard 150 additionally includes a vertical circuit board 163 supporting a USB port for programming a microprocessor, and an SD card for portable memory. The min-USB port also accepts a mini-USB adapter cable that supplies power from a wall-mounted AC adaptor. The AC adaptor is used, for example, when measurements are made over an extended period of time that exceeds the battery's life, or to recharge the battery 160. A Bluetooth transmitter 168 is mounted directly on the circuit board 150 and, following a measurement, wirelessly transmits information to an external monitor.

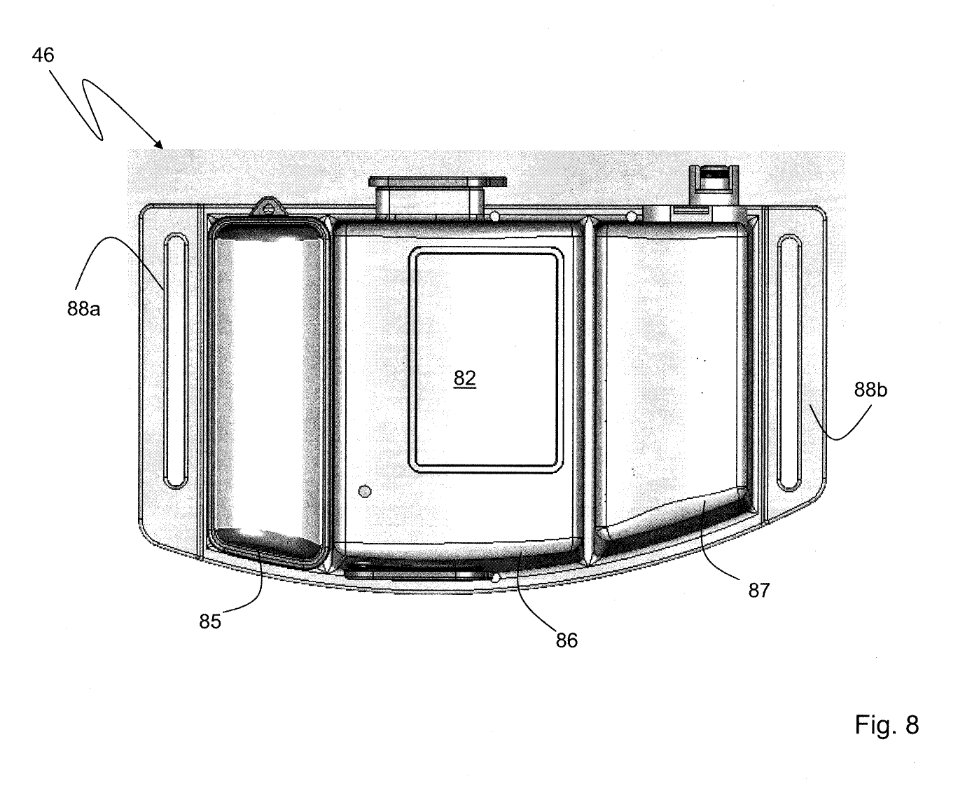

A flexible rubber housing 82, shown in FIG. 8, covers all the electronic components shown in FIG. 7. The housing 82 is divided into three separate compartments 85, 86, 87 covering, respectively, the battery 160, motherboard 150, and pneumatic components 165. On either side of the housing 82 a D-ring opening 88a, 88b receives a Velcro strap that connects to the armband 35, as described in FIG. 1. The housing 82 is typically formed from a polymeric flexible rubber which is relatively unaffected by heat, moisture, and sunlight. Alternatively, the housing 82 can be made of hard plastic with compartments that are joined by a hinged crease with electrical connections embedded into the hard plastic.

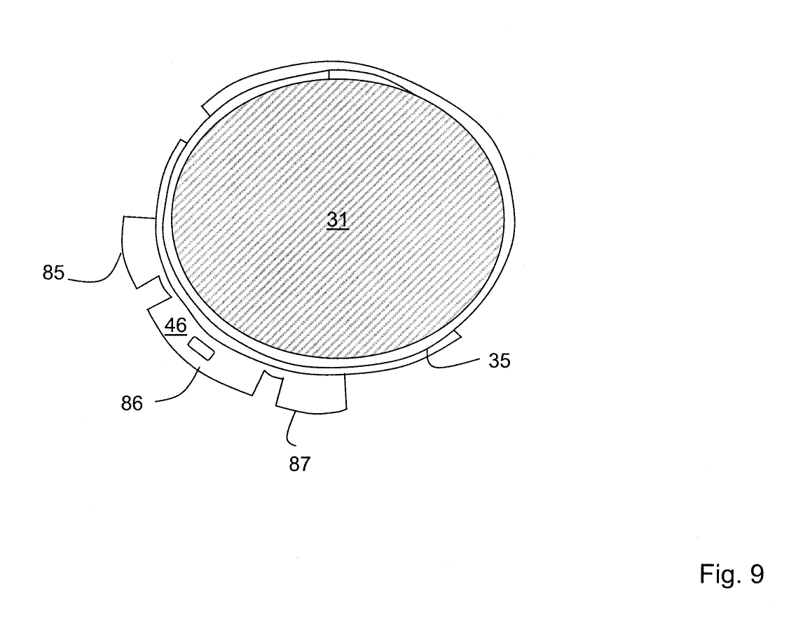

FIG. 9 shows a cross-sectional view of the body sensor 46 wrapping around the curvature of the patient's arm 31 and connected to the armband 35 that includes an inflatable bladder. The body sensor 46 and armband 35 are joined together by two D-ring connectors and Velcro straps (not shown in figure). Collectively the divided compartment 85, 86, 87 form a flexible housing that easily bends around the patient's arm 31. In this configuration the body sensor 46, which is centrally located on the arm 31, connects to the optical sensor on the thumb and to the ECG electrodes worn on the chest with minimal cable clutter, as described above.

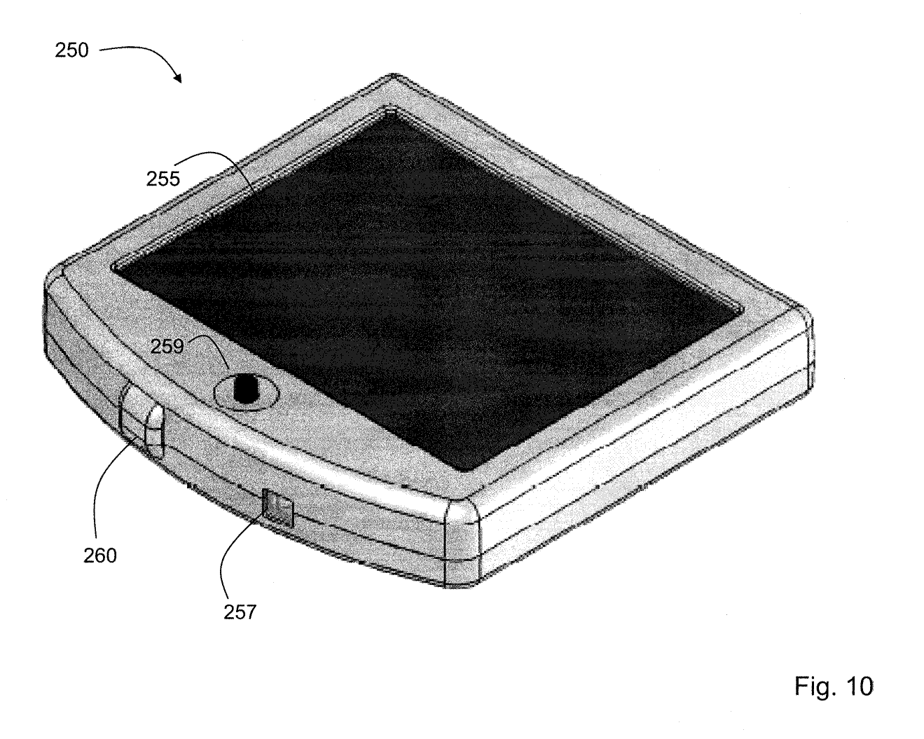

FIG. 10 shows a three-dimensional plan view of the monitor 250 that receives the Bluetooth-transmitted information from the body sensor. A front face of the monitor 250 includes a touchpanel display 255 that renders an icon-driven graphical user interface, and a circular on/off button 259. During an actual measurement, the touchpanel display 255 renders vital sign information from the body sensor. Such a monitor has been described previously in the following co-pending patent application, the contents of which are fully incorporated herein by reference: BLOOD PRESSURE MONITOR (U.S. Ser. No. 11/530,076; filed Sep. 8, 2006) and MONITOR FOR MEASURING VITAL SIGNS AND RENDERING VIDEO IMAGES (U.S. Ser. No. 11/682,177; filed Mar. 5, 2007).

The monitor 250 includes an internal Bluetooth transmitter (not shown in the figure) that includes an antenna 260 to increase the strength of the received signal. To pair with a body sensor, such as that shown in FIG. 9, the monitor 250 includes a barcode scanner 257 on its top surface. During operation, a user holds the monitor 250 in one hand, and points the barcode scanner 257 at a printed barcode adhered to the plastic cover surrounding the body sensor. The user then taps an icon on the touchpanel display 255, causing the barcode scanner 257 to scan the barcode.

The printed barcode includes information on the body sensor's Bluetooth transceiver that allows it to pair with the monitor's Bluetooth transceiver. The scanning process decodes the barcode and translates its information to a microprocessor within the monitor 250. Once the information is received, software running on the microprocessor analyzes it to complete the pairing. This methodology forces the user to bring the monitor into close proximity to the body sensor, thereby reducing the chance that vital sign information from another body sensor is erroneously received and displayed. The above-described system can be used in a number of different settings, including both the home and hospital.

In addition to those methods described above, a number of additional methods can be used to calculate blood pressure from the PPG and ECG waveforms. These are described in the following co-pending patent applications, the contents of which are incorporated herein by reference: 1) CUFFLESS BLOOD-PRESSURE MONITOR AND ACCOMPANYING WIRELESS, INTERNET-BASED SYSTEM (U.S. Ser. No. 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) VITAL SIGN MONITOR FOR ATHLETIC APPLICATIONS (U.S. Ser. No. ; filed Sep. 13, 2004); 5) CUFFLESS BLOOD PRESSURE MONITOR AND ACCOMPANYING WIRELESS MOBILE DEVICE (U.S. Ser. No. 10/967,511; filed Oct. 18, 2004); 6) BLOOD PRESSURE MONITORING DEVICE FEATURING A CALIBRATION-BASED ANALYSIS (U.S. Ser. No. 10/967,610; filed Oct. 18, 2004); 7) PERSONAL COMPUTER-BASED VITAL SIGN MONITOR (U.S. Ser. No. 10/906,342; filed Feb. 15, 2005); 8) PATCH SENSOR FOR MEASURING BLOOD PRESSURE WITHOUT A CUFF (U.S. Ser. No. 10/906,315; filed Feb. 14, 2005); 9) PATCH SENSOR FOR MEASURING VITAL SIGNS (U.S. Ser. No. 11/160,957; filed Jul. 18, 2005); 10) WIRELESS, INTERNET-BASED SYSTEM FOR MEASURING VITAL SIGNS FROM A PLURALITY OF PATIENTS IN A HOSPITAL OR MEDICAL CLINIC (U.S. Ser. No. 11/162,719; filed Sep. 9, 2005); 11) HAND-HELD MONITOR FOR MEASURING VITAL SIGNS (U.S. Ser. No. 11/162,742; filed Sep. 21, 2005); 12) CHEST STRAP FOR MEASURING VITAL SIGNS (U.S. Ser. No. 11/306,243; filed Dec. 20, 2005); 13) SYSTEM FOR MEASURING VITAL SIGNS USING AN OPTICAL MODULE FEATURING A GREEN LIGHT SOURCE (U.S. Ser. No. 11/307,375; filed Feb. 3, 2006); 14) BILATERAL DEVICE, SYSTEM AND METHOD FOR MONITORING VITAL SIGNS (U.S. Ser. No. 11/420,281; filed May 25, 2006); 15) SYSTEM FOR MEASURING VITAL SIGNS USING BILATERAL PULSE TRANSIT TIME (U.S. Ser. No. 11/420,652; filed May 26, 2006); 16) BLOOD PRESSURE MONITOR (U.S. Ser. No. 11/530,076; filed Sep. 8, 2006); 17) TWO-PART PATCH SENSOR FOR MONITORING VITAL SIGNS (U.S. Ser. No. 11/558,538; filed Nov. 10, 2006); and, 18) MONITOR FOR MEASURING VITAL SIGNS AND RENDERING VIDEO IMAGES (U.S. Ser. No. 11/682,177; filed Mar. 5, 2007).

Other embodiments are also within the scope of the claims. For example, other techniques, such as conventional oscillometry, can be used to determine systolic blood pressure for the above-described algorithms.

In other embodiments, a variety of software configurations can be run on the monitor to give it a PDA-like functionality. These include, for example, Micro C OS®, Linux®, Microsoft Windows®, embOS, VxWorks, SymbianOS, QNX, OSE, BSD and its variants, FreeDOS, FreeRTOX, LynxOS, or eCOS and other embedded operating systems. The monitor can also run a software configuration that allows it to receive and send voice calls, text messages, or video streams received through the Internet or from the nation-wide wireless network it connects to. The barcode scanner described with reference to FIG. 10 can also be used to capture patient or medical professional identification information, or other such labeling. This information, for example, can be used to communicate with a patient in a hospital or at home. In other embodiments, the device can connect to an Internet-accessible website to download content, e.g., calibrations, software updates, text messages, and information describing medications, from an associated website. As described above, the device can connect to the website using both wired (e.g., USB port) or wireless (e.g., short or long-range wireless transceivers) means. In still other embodiments, ‘alert’ values corresponding to vital signs and the pager or cell phone number of a caregiver can be programmed into the device using its graphical user interface. If a patient's vital signs meet an alert criteria, software on the device can send a wireless ‘page’ to the caregiver, thereby alerting them to the patient's condition. For additional patient safety, a confirmation scheme can be implemented that alerts other individuals or systems until acknowledgment of the alert is received.

Still other embodiments are within the scope of the following claims.

What is claimed is:

1. A system for measuring a blood pressure value from a patient, said system comprising:
a sensor configured to be worn on the patient's thumb comprising a light source that emits optical radiation, and a photodetector that detects the optical radiation after passing through a portion of a vessel in the patient's thumb to generate a first time-dependent signal;
at least two electrodes configured to be worn on the patient's body and detect electrical signals, each electrode connected to an electrical circuit configured to generate a second time-dependent signal; and
a processing system configured to be worn on a portion of the patient's arm and collectively process both the first and second time-dependent signals to determine the blood pressure value, the processing system attached to a housing comprising a first input port that receives the first time-dependent signal, or a signal used to generate the first time-dependent signal, and a second input port that receives the second time-dependent signal, or a signal used to generate the second time-dependent signal, the housing comprising the first input port on one side portion and the second input port on a second side portion opposite to the first side portion.

2. The system of claim 1, wherein the processing system comprises at least two separate portions, both configured to be worn on a separate portion of the patient's arm.

3. The system of claim 2, wherein a first portion comprises a processor configured to process the first and second time-dependent signals to determine the blood pressure value.

4. The system of claim 3, wherein the processor is further configured to process a time difference separating a first feature of the first time-dependent signal, and a second feature of the second time-dependent signal, and determine the blood pressure value therefrom.

5. The system of claim 4, wherein a second portion comprises a pneumatic system.

6. The system of claim 5, wherein the pneumatic system comprises a pump, a valve, and a manifold.

7. The system of claim 6, further comprising an inflatable armband configured to attach the pneumatic system to a portion of the patient's arm.

8. The system of claim 5, wherein the housing comprises both the first and second portions.

9. The system of claim 8, wherein the housing comprises a first compartment to house the first portion, and a second compartment to house the second portion, the first and second portions configured to attach to separate portions of the patient's arm.

10. The system of claim 9, wherein the first and second portions are separated and connected through a flexible cable.

11. The system of claim 9, wherein the housing comprises a flexible material.

12. The system of claim 11, wherein the housing is further configured to wrap around a portion of the patient's aim.

13. The system of claim 5, wherein the pneumatic system is configured to supply a pressure to the patient's arm, and the processor is further configured to process the first time-dependent signal in the presence of the supplied pressure to determine a blood pressure calibration.

14. The system of claim 13, wherein the processor is further configured to process the time difference in the presence of the supplied pressure to determine a blood pressure calibration.

15. The system of claim 14, wherein the processor is further configured to process an increase in the time difference as a function of pressure to determine the blood pressure calibration.

16. The system of claim 13, wherein the processor is further configured to process a decrease in an amplitude of the first time-dependent signal to determine the blood pressure calibration.

17. The system of claim 1, wherein the sensor configured to be worn on the patient's thumb comprises a flexible component configured to wrap around a portion of the patient's thumb while leaving a tip of the thumb uncovered.

18. The system of claim 17, wherein the flexible component is configured to wrap around a base of the patient's thumb.

19. The system of claim 18, wherein the flexible component comprises at least two light sources and a photodetector.

20. The system of claim 1, further comprising a wireless transmitter.

21. The system of claim 20, further comprising an external display configured to receive information from the processing system through the wireless transmitter.

22. A system for measuring a blood pressure value from a patient, comprising:
a sensor configured to be worn on the patient's thumb comprising a light source that emits optical radiation, and a photodetector that detects the optical radiation after passing through a portion of a vessel in the patient's thumb to generate a first time-dependent signal;
at least two electrodes configured to be worn on the patient's body and detect electrical signals, each electrode connected to an electrical circuit configured to generate a second time-dependent signal;
a pneumatic system comprising a pump, a valve, and a manifold, the pneumatic system configured to supply a pressure to the patient's arm; and
a processing system configured to be worn on a portion of the patient's arm and collectively process both the first and second time-dependent signals in the presence of pressure supplied by the pneumatic system to determine the blood pressure value, the processing system comprising a housing comprising a first input port that receives the first time-dependent signal, or a signal used to generate the first time-dependent signal, and a second input port that receives the second time-dependent signal, or a signal used to generate the second time-dependent signal, the housing comprising the first input port on one side portion and the second input port on a second side portion opposite to the first side portion.

23. A system for measuring a blood pressure value from a patient, comprising:
a sensor configured to be worn on the patient's thumb comprising a light source that emits optical radiation, and a photodetector that detects the optical radiation after passing through a portion of a vessel in the patient's thumb to generate a first time-dependent signal;
at least two electrodes configured to be worn on the patient's body and detect electrical signals, each electrode connected to an electrical circuit configured to generate a second time-dependent signal;
a pneumatic system comprising a pump, a valve, and a manifold, the pneumatic system configured to supply a pressure to the patient's arm and generate a third time-dependent signal representing the pressure; and
a processing system configured to be worn on the patient's arm and comprising: i) a first compartment comprising the pneumatic system configured to be worn on a first portion of the patient's arm; and ii) a second compartment, connected to the first compartment through a flexible cable, and configured to be worn on a second portion of the patient's arm, the second compartment comprising a processor that receives the first time-dependent signal, or a signal used to generate the first time-dependent signal, the second time-dependent signal, or a signal used to generate the second time-dependent signal, and the third time-dependent signal, or a signal used to generate the third time-dependent signal, the processor configured to determine a blood pressure value from the first, second, and third time-dependent signals.
